(19) United States
(12) Patent Application Publication
(10) Pub. No.: US 2011/0015696 A1
(43) Pub. Date: Jan. 20, 2011

(54) Adaptive Muscle Stimulation Apparatus and Methods

(76) Inventor: Larry Joseph Kirn, Austin, TX (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/838,071

(22) Filed: Jul. 16, 2010

Related U.S. Application Data
(60) Provisional application No. 61/226,167, filed on Jul. 16, 2009.

(51) Int. Cl. A61N 1/36

(52) U.S. Cl. 607/48

(57) ABSTRACT

A portable device, with requisite method, is used to support a compromised joint, such as by arthritis, through dynamic stimulation of the surrounding musculature. Direct feedback, preferably from the patient wearing the device, is used during problematic joint operation to qualify dynamic measured or calculated spacial or physical conditions of the compromised joint for use as state definitions. Approximation of these state definitions by subsequent spacial or physical conditions of the compromised joint evokes variable stimulation of the surrounding musculature. State definitions so qualified, as well as control of stimulation output, may include magnitude and/or vector of joint force, facilitating predictable benefit from the device beyond that available through positional control.

### REFERENCE TO RELATED APPLICATION

This application claims priority from U.S. Provisional Patent Application Ser. No. 61/226,167, filed Jul. 16, 2009, the entire content of which is incorporated herein by reference.

### FIELD OF THE INVENTION

This invention relates generally to medical devices, and particularly to apparatus and methods to mitigate and/or rehabilitate compromised skeletal joints through the use of dynamic muscle stimulation which responds both to patient demand and activity.

### BACKGROUND OF THE INVENTION

Pathology or injury involving a joint has longer-term consequences than many other medical conditions. Especially since mobility and physical function is possibly chronically affected, a great deal of attention has been paid to mitigating the effects of compromised joints. External bracing usually is minimally effective, due to the necessary imposition of soft tissue between a rigid brace and rigid skeletal elements. It has been noted that the direct connection of the body's own musculature to these skeletal elements can be used advantageously for support; no external device is so intimately connected to the skeleton as the body's own musculature. Both electrical and magnetic stimulation have potential benefit to this end. Constant firing of this musculature holds extremely limited benefit; controlled firing appropriate to physical function has been shown to be necessary. The specific instances at which muscles must be fired in order to have protective effect on a compromised joint, however, are usually entirely different than muscle control learned prior to the anomaly. Retraining conscious muscle control to compensate for an acquired abnormality is therefore usually unsuccessful. Both electrical and magnetic stimulation of muscles have had success in clinical settings when used to strengthen or retrain muscles surrounding a compromised joint. Arthritic knees, which respond poorly to external bracing, are a ready candidate for such support. U.S. Pat. Nos. 6,659,918 and 7,163,492, for example, teach use of muscle stimulation in conjunction with clinical exercise equipment for this purpose.

Muscle stimulation devices used in this setting, however, have given limited and impermanent results. These devices employ fixed movement patterns which the patient must follow, limiting immediate benefit to the activity imposed by the specific exercise machine. Although benefit from muscle strengthening has repeatedly been shown from their use, these devices often fail to retrain the patients' use of the target muscles, presumably due to marked differences between the fixed exercise activity and non-clinical ambulant movements.

In search of more permanent solutions usable outside clinical settings, dynamic control of muscle stimulation devices has been investigated. U.S. Pat. Nos. 4,569,352, 4,796,631, and 6,507757 all teach use of angular limb position, possibly in conjunction with foot contact force detection, to digitally control application of muscle stimulation. Although usable in ambulant settings, poor correlation between absolute limb position, or heel loading, and patient function limits efficacy to a very limited set of motions. This poor correlation results both from inertial joint force components that may be relatively independent of limb position, and from the digital (on/off) control used. The assumption in these implementations that heel contact establishes gravitic force through a leg fails to address the broad range of force through a limb as a patient goes through everyday activities, as well as the fact that identical force vectors may be exerted on a joint from a multitude of physical positions. Forces exerted on a joint are comprised of both gravitic and inertial elements, an extreme example of which is a rapid direction change by a soccer player. Support provided by simple digital on/off stimulation control cannot effectively counter-balance this broad range of force applied to the joint.

Inclusion of gait cycle information, as a motion or position template, as been somewhat successful in improving ambulant control of muscle stimulators over simple position and/or heel strike detection. U.S. Pat. Nos. 5,814,093 and 5,643,332, for example, teach comparison between measured leg angular position in time and stored template values to digitally control muscle stimulation. Several factors frustrate even template-driven stimulation control, however. Not only do all patients present deviations from an idealized gait cycle, they each present broad variation within each phase of gait, under influence of many external variables. Environmental conditions, clothing, sensor positions, specific movements of an action, and motion speed are among these many variables, but the set as well includes difficult inputs such as mood, weight of items being carried, etc. Movement variability, even in a specific patient, is therefore so broad as to cause significant overlap between advantageous stimulation and non-stimulation states, negatively impacting operation. Another fundamental difficulty with positional control is that the external force incident on the joint, being transferred or supported by the stimulated muscle, is itself highly variable, and not deterministic with position. Different activities cause a wide range of both force amplitudes and vectors in limbs, even when occurring at identical inclinations from the earth.

Although need has driven the majority of dynamic muscle stimulation to knee applications, the fact that the knee comprises two load-bearing surfaces additionally is not broadly addressed. Force vectors of the two load-bearing condyles of the knee are usually related, but the differential force magnitude experienced between them in normal ambulant activities is not within the scope of simple leg positional control, even when heel force is taken into account. The majority of degraded knees have damage to a single bearing surface, and advantageous offloading of force from that degraded condyle to the intact bearing surface through firing contralateral musculature is fundamental to many applications of this technology. Determination of all joint forces is necessary in order to determine appropriate stimulation for effective force offloading.

None of the preceding solutions, however, address the fact that the optimum functional feedback for an ambulant device supporting a joint is often pain, and that the most authoritative indicator of pain is usually the patient. Direct patient input to functional state definitions, however, is not found in practice.

A need exists for an apparatus and method whereby supplementary enlistment through stimulation of musculature around a compromised joint or joint element may be directly responsive to patient feedback and that both vector and amplitude of force exerted on that joint or joint element may be included in the information used to control muscle stimulation.

### SUMMARY OF THE INVENTION

The present invention resides in the technique of utilizing qualifying feedback, primarily from the patient, to identify and store measured and/or calculated physical conditions precipitating instances of problematic operation of a compromised joint; comparing this stored information with real-time measured and/or calculated physical conditions to detect impending or current problematic joint operation, and stimulating surrounding musculature as a function of one or more measured and/or calculated physical conditions while existing conditions approximate those indicating problematic joint operation. Physical conditions used may include both magnitude and vector of forces exerted upon the compromised joint

A method for supporting a compromised joint through dynamic stimulation of surrounding musculature comprising the steps of:
1. Measuring and/or calculating at least one spacial or physical condition of the compromised joint.
2. Dynamically identifying instances of problematic joint operation, preferably involving patient input.
3. Storing state definitions comprised of spacial or physical conditions of the compromised joint during identified instances of problematic joint operation.
4. Comparing ongoing spacial or physical conditions of the compromised joint against stored state definitions of problematic joint operation.
5. Stimulating surrounding musculature of the compromised joint to elicit support during periods of time that spacial or physical conditions of the compromised joint approximate stored state definitions.

### BRIEF DESCRIPTION OF THE DRAWINGS

FIG. 1 shows a block diagram of a preferred embodiment of the present invention.

### DETAILED DESCRIPTION OF THE INVENTION

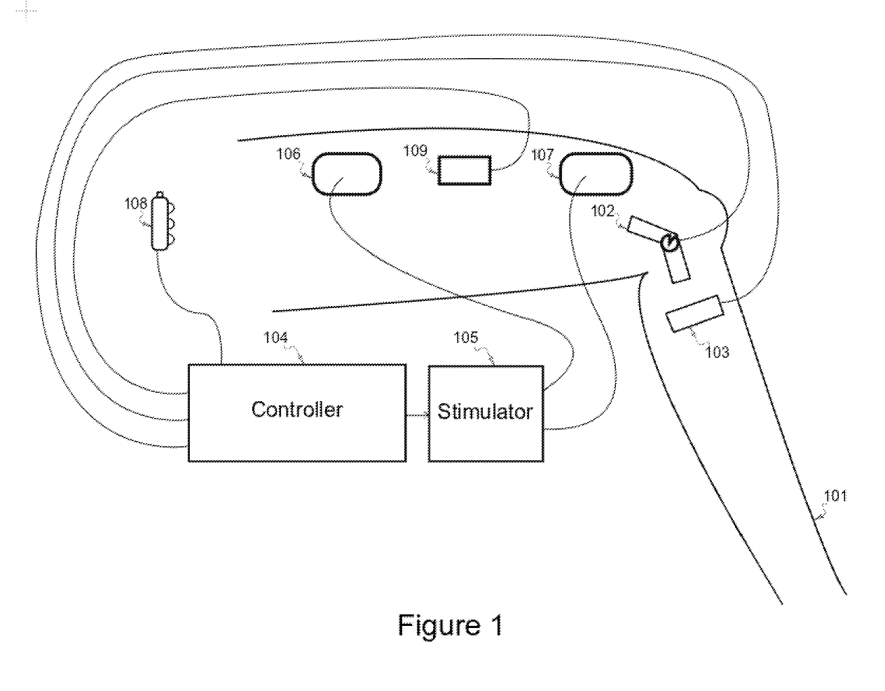

Referring now to FIG. 1, Knee Position Sensor 102, Inclinometer 103, and optionally Muscle Contraction Sensor 109 are affixed to a human Leg 101. Knee Position Sensor 102 may be embodied variously as a variably resistive or optical flexion sensor, differential multi-axis accelerometers, differential gyros, or any other variant of positional or differential positional transducer, as are used in the art. Inclinometer 103 may also be implemented with a number of sensor types, possibly included as a function of Knee Position Sensor 102. Outputs of sensors 102, 103, and 109 are supplied as input to Controller 104, which uses said sensor outputs to ascertain movements and function of Leg 101, as well as magnitudes and vectors of forces upon components of Leg 101. Controller 104 as well receives the output of Switch 108. Controller 104, under conditions defined below, outputs a variable control signal to Muscle Stimulator 105, which in response emits high-voltage pulses to transcutaneous Electrodes 106 and 107. Said Electrodes 106 and 107 are attached to Leg 101 over the specific muscle from which additional joint support is desired. In response to said high-voltage pulses through Electrodes 106 and 107, the underlying muscle will contract in rough proportion to the current integral of said high-voltage pulses, as is known in the art. Optional Muscle Contraction Sensor 109 is fastened to Leg 101 directly upon the muscle to be so fired by Electrodes 106 and 107, and provides to Controller 104 a feedback signal denoting relative force of muscle contraction elicited by said Stimulator 105. Note that, for sake of simplicity, fewer transcutaneous electrodes and/or sensors are shown than may be required in any given implementation. Note further that Muscle Contraction Sensor 109 serves to optimize performance, but is not fundamental to the present invention.

In operation, the patient wearing the invention on Leg 101 will perform motions in the course of daily activity during which additional muscle support is necessary, and press Switch 108 when pain or other undesirable conditions which may be mitigated from supplementary muscle support occur. Upon receipt of this signal from Switch 108, Controller 104 determines precipitating movements and vectored forces, through sensor means described above, and stores this precipitating data as a Muscle Support Event, possibly including the amount of compensatory muscle force necessitated by the precipitating forces. Muscle Support Events, rather than template definitions of prior art, therefore form state definitions under which muscle stimulation is to be performed.

Repetitive iteration of problematic motions with Switch 108 activation during each iteration may be used, to allow Controller 104 to better qualify Muscle Support Events, using any of the averaging or statistical approaches known to adaptive control art. Switch 108 may be permanently or temporarily connected to Controller 104, either directly or through any indirect communication means, such as a radio-frequency signal.

The data comprising each Muscle Support Event so qualified through Switch 108 is stored in the memory of Controller 104 for subsequent use. It is as well assumed that Controller 104 utilizes adaptive techniques known to the art, to functionally normalize all inputs on an ongoing basis. This is necessary to compensate the expected variances of clothing, environment, fatigue, etc., and to normalize Muscle Support Events, which will be identified at differing times, with each other.

In subsequent operation, Controller 104, upon recognition of conditions, possibly including force vectors and/or magnitudes calculated from physical inputs, conforming to any of said stored Muscle Support Events, emits a control signal to Stimulator 105, so as to induce muscle contraction through the techniques and apparatus described above. Output parameters, including magnitude, of said muscle contraction control signal may be determined in part by both stored requisite force in the Muscle Support Event and calculated joint force determined by sensor inputs. Upon determination by Controller 104 that the active Muscle Support Event has terminated, signals to Stimulator 105 cease.

Note that forces on a joint in ambulant situations consist of both gravitic and inertial components. Force vectors and magnitudes defined within a qualified Muscle Support Event may therefore be precipitated by and subsequently approximated by multiple limb positions or velocities, due to the variable contribution of the two force components. Incorporation of force, as opposed to simple position, in state definitions therefore facilitates protective support by the invention in physical orientations beyond those in which the Muscle Support Event was originally identified. Muscles contralateral to each element of joints possessing multiple bearing surfaces may require independently-controlled stimulation appropriate to counteract forces incident on each joint element. This may be achieved through multiple instances of appropriate elements of the invention, as will be seen by one skilled in the art.

It is known that the force of muscle contractions under extrinsic stimulation is only very loosely related to the magnitude of stimulation. The duration of stimulated contractions are also strongly affected by runaway spasms which can be induced by unregulated stimulation. To nullify these variabilities, feedback from Contraction Sensor 109 is expected to be used by Controller 104 in most embodiments, to create controlled forces and enhance patient comfort.

Note that Muscle Support Events include data calculated by the controller that may be indirectly coupled to sensor inputs or extrapolative in nature; inference, such as of inertial components, etc., may or may not be included in these events. Sensors initially anticipated include accelerometers, resistive, magnetic, inertial, and capacitive devices, but as well are expected to possibly include larger-scale systems, such as Global Position Sensing.

Although transcutaneous conductive electrical stimulation is shown to effect muscle firing, alternative techniques, such as magnetic, inductive, or capacitive stimulation, are as well anticipated.

The variability problems normally seen in fixed or adjustable template-driven stimulation control systems can be seen by the disclosure above to be ameliorated by the use of qualified event-driven state definitions which more closely replicate external forces incident on a compromised joint. Inclusion of forces incident on the joint in both state definitions and output control serves to better stabilize a compromised joint against external forces applied. Resultantly, a system using the techniques described herein can elicit skeletal support of a compromised joint by the connected musculature in an extremely predictable and comfortable fashion.

I claim:

1. A system for supporting a compromised joint through dynamic stimulation of surrounding musculature comprising:
means to measure and/or calculate at least one spacial or physical condition of at least one skeletal element connected to said compromised joint;
means to dynamically indicate instances of problematic operation of said compromised joint;
means to store state definitions comprised of at least one said spacial or physical condition of said compromised joint during said instances of problematic operation;
means to recognize approximation of said spacial or physical conditions of said compromised joint to any of said state definitions; and
means to stimulate surrounding musculature during periods of time in which said spacial or physical conditions of said compromised joint approximate any of said state definitions.

2. The system of claim 1 wherein at least one said spacial or physical condition of said compromised joint includes force exerted on said compromised joint.

3. The system of claim 1 wherein said means to identify instances of problematic operation comprises a directly-connected switch.

4. The system of claim 1 wherein said means to identify instances of problematic operation is connected wirelessly.

5. The system of claim 1 wherein said means to measure and/or calculate at least one spacial or physical condition includes an accelerometer.

6. The system of claim 1 wherein said means to stimulate surrounding musculature comprises an electrical stimulator.

7. The system of claim 1 wherein one or more comprised element is multiply embodied for each element of a compromised joint possessing multiple bearing surfaces.

8. The system of claim 1 wherein one or more output characteristics of said means to stimulate surrounding musculature is a function of one or more of said spacial or physical conditions of said compromised joint.

9. The system of claim 1 wherein said compromised joint is the knee.

10. The system of claim 1 wherein said compromised joint is a joint other than the knee.

11. The system of claim 1 wherein said system includes software executing on a processing unit.

12. A method for supporting a compromised joint through dynamic stimulation of surrounding musculature comprising the steps of:
measuring and/or calculating at least one spacial or physical condition of at least one skeletal element connected to said compromised joint;
dynamically identifying instances of problematic operation of said compromised joint;
storing at least one said spacial or physical condition of said compromised joint during said instances of problematic operation as state definitions;
comparing said measured or calculated spacial or physical conditions of said compromised joint with said state definitions stored during said instances of problematic operation; and
stimulating said surrounding musculature of said compromised joint during periods of time in which said measured or calculated spacial or physical conditions of said compromised joint approximate one or more said state definition stored during said instances of problematic operation.

13. The method of claim 12 wherein identification of said instances of problematic operation of said compromised joint is initiated by the patient wearing the invention.

14. The method of claim 12 wherein identification of said instances of problematic force on said compromised joint is initiated by medical staff attending the patient wearing the invention.

15. The method of claim 12 wherein at least one said spacial or physical condition of said compromised joint includes magnitude and/or vector of gravitic force exerted upon said compromised joint.

16. The method of claim 12 wherein at least one said spacial or physical condition of said compromised joint includes magnitude and/or vector of inertial force exerted upon said compromised joint.

17. The method of claim 12 wherein one or more comprised step is multiply performed for each element of a compromised joint possessing multiple bearing surfaces.

18. The method of claim 12 whereby statistical averaging of said spacial or physical conditions, during multiple iterations of a movement causing said instances of problematic operation, is performed before storage.

19. The method of claim 12 whereby one or more output characteristics of said stimulating surrounding musculature is a function of one or more said spacial or physical condition of said compromised joint.

20. The method of claim 12 whereby one or more output characteristics of said stimulating surrounding musculature is a function of measured or calculated muscle contraction.
